(19) United States
(12) Patent Application Publication
(10) Pub. No.: US 2005/0233450 A1
(43) Pub. Date: Oct. 20, 2005

(54) Methods of inducing hair growth

(76) Inventors: Paul F. Goetinck, Boston, MA (US); Tokuro Iwabuchi, Yokohama (JP)

Correspondence Address: FISH & RICHARDSON PC, P.O. BOX 1022, MINNEAPOLIS, MN, 55440-1022, US

(21) Appl. No.: 11/035,287

(22) Filed: Jan. 12, 2005

Related U.S. Application Data
(60) Provisional application No. 60/535,842, filed on Jan. 12, 2004.

(51) Int. Cl. C12N 5/08

(52) U.S. Cl. 435/371

(57) ABSTRACT

This disclosure features methods and compositions for stimulating hair growth and for culture DP cells.

### CROSS-REFERENCE TO RELATED APPLICATION

This application claims priority to U.S. patent application Ser. No. 60/535,842, filed on Jan. 12, 2004, the entire contents of which are incorporated by reference.

### BACKGROUND

The hair follicle undergoes a cycle of hair growth (anagen) followed by regression (catagen), and quiescence (telogen) until a new hair shaft is generated in the existing follicle during the subsequent anagen phase (Hardy et al. (1992) Trends Genet. 8:55-61). The hair shaft is derived from the epithelial matrix cells at the base of the follicle, but a cluster of dermal cells ensheathed by the matrix cells, known as the dermal papilla (DP), is thought to supply inductive signals required for hair outgrowth.

The isolation of dermal papillae (DP) from human hair follicles and the establishment of primary cell cultures from the papilla explants in a nutrient medium have been reported (Messenger (1984) British J. of Dermatol. 110:685-689). Cultured rat vibrissa DP cells support dense hair growth in the nude mouse graft model for testing the hair-forming ability of selected cell populations when early passage DP cells are used. However, the DP cells progressively lose this hair inductive potential after repeated passage in culture (Lichti et al. (1993) J. Invest. Dermatol. 101:124S-129S; U.S. Application No. 20020114772).

### SUMMARY OF THE INVENTION

The inventors have found that the hair-inductive activity of a dermal papilla (DP) cell that has lost hair-inductive ability in culture, or has reduced hair-inductive ability in culture, e.g., as compared to in vivo, can be increased (e.g., reconstituted), or that the hair inductivity of a DP cell can be maintained, or its loss minimized, by culturing the DP cell under the appropriate conditions, e.g., in a manner that allows the rounding-up and/or self-aggregation of DP cells, or by culturing DP cells on a non-adherent substrate.

Accordingly, in one aspect, the invention is directed to methods of culturing a DP cell or DP cells derived from a mammal, e.g., a mouse, rat, sheep, dog, or primate, e.g., a human, chimpanzee, or monkey. In one embodiment, the method involves culturing the DP cell or DP cells on a non-adherent substrate. The non-adherent substrate is preferably an untreated (e.g., it has not been treated to increase adherence of cells) cell culture vessel, e.g., a dish, plate, or flask, e.g., a spinner flask. Alternatively, the non-adherent substrate is treated with a non-adherent compound, e.g., poly-HEMA (e.g., poly-2-hydroxyethyl methacrylate). Untreated plastic and other polymers can be used to provide a hydrophobic surface that is non-adherent.

In other embodiments, the method involves culturing DP cells under conditions that allow the DP cells to round up or to self-aggregate, e.g., to form spheroids. The DP cells can be cultured, e.g., for at least 6 hours, e.g., at least 12, 24, or 48 hours, or until they have increased or regained hair inductive activity. The method can also be used for other cells that modulate or participate in hair growth and for other dermal cells, including, for example, precursors of dermal papilla cells.

In a preferred embodiment the DP cells can have lost or reduced hair inductive activity. In other preferred embodiments the DP cells have not lost hair inductive activity.

In a preferred embodiment the cells are cultured until at least 10, 20, 30, 50, 60, 70, or 80% of the cells (preferably live cells) in the culture are non-adherent.

In a preferred embodiment the cells are cultured until at least 10, 20, 30, 50, 60, 70, or 80% of the cells (preferably live cells) in the culture are rounded up or self aggregated.

In a preferred embodiment the DP cell is cultured for at least 3, 6, 12, or 24 hours. In a preferred embodiment the cells are cultured for less than 48, 72, or 96 hours.

In one embodiment, the DP cells are at least 10, 20, 30, 50, 60, 70, 80, 90, or 99% pure, e.g., relative to other cells that may be present in the culture.

In another aspect, DP cells (e.g., DP cells derived from a human) are cultured and expanded on an adherent substrate, e.g., at least until the DP cells have lost hair inductive activity, e.g., at least 1 or 2 days, e.g., at least 5, 7, 10, 14, 28, or more days, and are thereafter cultured under a second set of conditions that allow the expanded DP cells to regain hair inductive activity, e.g., by culturing on a non-adherent substrate as described herein or by culturing under conditions described herein that allow the DP cells to self-aggregate or round up.

In a preferred embodiment the DP cells can have lost or reduced hair inductive activity at the end expansion culture.

In a preferred embodiment at least 10, 20, 30, 50, 60, 70, or 80% of the cells (preferably live cells) in the culture are non-adherent under the second set of conditions.

In a preferred embodiment at least 10, 20, 30, 50, 60, 70, or 80% of the cells (preferably live cells) in the culture are rounded up or self aggregated under the second set of conditions.

In a preferred embodiment the DP cell is cultured under the second set of conditions for at least 3, 6, 12, or 24 hours. In a preferred embodiment the cells are cultured under the second set of conditions for less than 48, 72, or 96 hours.

In another aspect, the invention features a cell culture of DP cells, e.g., a non-adherent, self-aggregated or rounded-up culture of DP cells as described herein. The DP cells of the culture have increased hair inductive activity compared to a monolayer culture of DP cells. In some embodiments, the culture includes a non-adherent substrate described herein.

In a preferred embodiment at least 10, 20, 30, 50, 60, 70, or 80% of the cells (preferably live cells) in the culture are non-adherent.

In a preferred embodiment at least 10, 20, 30, 50, 60, 70, or 80% of the cells (preferably live cells) in the culture are rounded up or self aggregated.

Another aspect of the invention relates to methods of stimulating hair growth in a subject, e.g., a human subject. The method includes culturing DP cells using a method described herein for reconstituting hair inductive activity and transplanting the cultured DP cells into the subject. In some embodiments, the method includes culturing and transplanting DP cells that are autologous, allogeneic, or xenogeneic to the subject. In some embodiments, the method includes transplanting the cultured DP cells onto the subject's skin, e.g., into a hair follicle in the subject's skin, e.g., scalp. In some embodiments, the method includes evaluating hair growth in the subject. The evaluation can be performed before and/or after the transplanting.

In another aspect, the invention features a pharmaceutical composition for stimulating hair growth in a subject that includes a DP cell made by a method described herein. In some embodiments, the pharmaceutical composition includes a carrier or excipient, e.g., saline. Another aspect relates to a pharmaceutical container, e.g., a microsyringe, including a pharmaceutical composition described herein. The container can be used in a method of stimulating hair growth in a subject. The container can include a non-adherent surface, e.g., part or all of the internal surface is non-adherent.

Another aspect features a kit for stimulating hair growth. The kit includes instructions for culturing DP cells according to the methods described herein and one or more of: a non-adherent cell culture vessel described herein, growth medium components, and a pharmaceutical container, such as a microsyringe, for transplanting cultured DP cells. In still another aspect, the invention features a cell culture vessel that includes a non-adherent surface, e.g., as described herien, a medium, and one or more DP cells. Exemplary vessels include microtubes, flasks, petri plates, microtitre plates, and so on.

The term “plated” or “plating” as used herein in reference to cells refers to establishing cell cultures in vitro. For example, cells can be diluted in cell culture media and then added to a cell culture vessel, e.g., a cell culture place, dish or flask. Cells can be plated at a variety of concentrations and/or cell densities.

“Cell passaging” refers to taking cells from a culture and dividing the cells into multiple cultures. “Cell passaging” adherent cells can involve the steps of (1) releasing cells from a solid support and disassociation of these cells, and (2) diluting the cells in fresh media suitable for cell proliferation. Alternatively, for cells grown on non-adherent substrates, cell passaging can involve diluting the cells in fresh media and dividing the cells into multiple cultures.

The terms “expand,” “expanded,” “expanding,” “proliferate,” and “proliferation,” as used herein in reference to cells, refers to culturing a group of cells so that cells increase in size and/or can increase in number over a period of time. Such a culture is referred to as an “expansion culture”. In preferred embodiments, cultured cells are expanded for at least 2, 5, 7, 10, 14, 28, or more days.

The terms “confluence” and “confluency” as used herein refer to a cell culture in which about more than 50% of the cells physically contact at least one other cell in the culture.

The term “culture” as used herein in refers to an in vitro environment in which cells are maintained and/or grown. The in vitro environment can include a vessel containing any routine medium that is suitable for maintaining cells in vitro, e.g., a suitable liquid medium. Specific examples of suitable in vitro environments for cell cultures are described in Culture of Animal Cells: a manual of basic techniques (3^{rd} edition), 1994, R. 1. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; and Animal Cells: culture and media, 1994, D. C. Darling, S. J. Morgan, John Wiley and Sons, Ltd., each of which is incorporated herein by reference in its entirety including all figures, tables, and drawings. Preferred medium is Dulbecco's Modified Eagle's Medium (DMEM, available from Gibco 10829-108).

Cells can be co-cultured in suspension and/or in monolayers with one or more substantially similar cells. Cells may be co-cultured in suspension and/or in monolayers with a heterogeneous (different) population of cells. The term “heterogeneous” can relate to any cell characteristic, e.g., a different cell type or cell cycle stage. In preferred embodiments, cells are co-cultured in conditions where they do not adhere to a solid support.

The term “non-adherent substrate” refers to a substrate that does not support the attachment of cells to its surface. The substrate is typically a cell culture vessel. Cells proliferating on a non-adherent substrate can be in suspension. Cells in suspension may be stirred while proliferating using routine techniques. Accordingly to be “cultured on a non-adherent substrate” refers to be cultured in a milieu which includes a non-adherent substrate, e.g., a substrate with which cells may making transient contact. The term “adherent substrate” refers to a substrate that does support the attachment of cells to its surface.

The term “monolayer” as used herein refers to cells that are attached to a solid support while proliferating in suitable culture conditions. A small portion of the cells proliferating in the monolayer under suitable growth conditions can be indirectly attached to the solid support through an attachment to other cells. Preferably fewer than 15% of the cultured cells are indirectly attached to the solid support in a monolayer, e.g., fewer than 10, 9, 8, 7, 6, or 5% of the cultured cells are indirectly attached to the solid support in a monolayer.

The term “isolate,” “isolated,” and “isolating,” when referring to a cell, means the physical separation of a subject cell from a group of cells, e.g., a tissue, e.g., a dermal papilla, a cell culture, a cell line, or an animal.

“Self-aggregation” and “self-aggregate” refer to the ability of cells to adhere to one another to form spheroids of cells. A “spheroid” is a group of cells in which at least 70% of cells are attached to other cells and are not directly attached to a substrate. A spheroid includes at least 2 cells and can include, e.g., 4, 8, 12, 20, 40, 50, 100, 500, 1000, or more cells.

“Rounded up” in reference to a cultured cell means that the cell is not attached to a substrate or to another cell. Such a cell typically appears as a round ball floating in the culture medium.

“Inductive” and “inductiveness” refer to the ability of a dermal papilla cell to cause hair outgrowth from a hair follicle. “Non-inductive” refers to cells not able to cause hair outgrowth.

“Transplanting” refers to inserting cells into a live subject. The cells can be obtained from another subject or the same subject.

The methods and compositions described herein can be used to reconstitute the hair inductive activity of DP cells that have lost hair inductive activity, e.g., during in vitro culture. The methods described herein allow a DP cell population to be significantly expanded in vitro and, if the hair inductive ability of the DP cells is lost during such expansion period, the hair inducing ability can be reconstituted prior to implantation. Such reconstituted DP cells can be used for implanting onto a subject's skin, e.g., scalp, to induce hair growth.

### DETAILED DESCRIPTION

Dermal papilla (DP) cells in vivo are known to stimulate hair growth in intact follicles. However, DP cells lose their hair-inducing capacity over time when cultured in vitro (Lichti et al. (1993) J. Invest. Dermatol. 101:27S-32S). The invention is based, at least in part, on the inventors' discovery of methods of restoring the hair inductive capacity of cultured DP cells after they have lost hair inductiveness in vitro. These methods are based on the inventors' discovery that culturing DP cells that have lost their hair inductive capacity in a manner that allows them to round up and/or self-aggregate, rather than to adhere to a substrate (e.g., by culturing the DP cells in a vessel with non-adherent surfaces), restores the ability of the DP cells to induce hair growth. Accordingly, the invention encompasses, among other things, DP cell cultures, methods for culturing DP cells, and methods and compositions for stimulating hair growth.

Dermal Papilla Cells

The dermal papilla (DP) is a cluster of mesenchymal dermal papilla cells that is ensheathed by matrix cells. The invention is not limited in the technique used to isolate DP cells from a DP. Such techniques can include, e.g., explanting a DP from a hair follicle, culturing the explant, and allowing the DP cells to emigrate from the DP explant into the culture (see, e.g., Messenger (1984) British J. of Dermatol. 110:685-689; Magerl et al. (2002) Exp. Dermatol. 11:381-385). Alternatively, DP cells can be removed from an explanted DP and cultured separately.

DP cells useful in the methods described herein can be derived from mammals, e.g., mice, rats, sheep, dogs, or primates, e.g., humans, chimpanzees, or monkeys. The DP cells can also be derived from transgenic animals, e.g., transgenic mice, and can include a suitable expression marker, e.g., green fluorescent protein (GFP). The expression of the marker can be used to determine the inductive activity of the DP cells, as described by Kishimoto et al. (2000) Genes and Development 14:1181-1185.

Methods of Culturing

Dermal papilla cells are isolated as described herein and are cultured in a culture medium using routine methods. A suitable medium can be, e.g., a commercially available medium such as Ham's F10 (Sigma), Minimal Essential Medium (MEM, Sigma), RPMI-1640 (Sigma), Iscove's Modified Dulbecco's Medium (IMDM), and Dulbecco's Modified Eagle's Medium (DMEM, Sigma). In addition, any of the media described in Ham and Wallace, (1979) Meth. Enz., 58:44; Barnes and Sato, (1980) Anal. Biochem. 102:255; U.S. Pat. Nos. 4,767,704; 4,657,866; 4,927,762; 4,560,655; or 5,122,469; WO 90/03430; WO 87/00195; or U.S. Pat. No. Re. 30,985 may be used as culture medium for the cells. In the methods described herein, DMEM is preferably used. The medium can be supplemented as necessary with serum (such as fetal bovine serum, calf serum, or horse serum), hormones and/or other growth factors (such as insulin, transferrin, or epidermal growth factor), ions (such as sodium, chloride, calcium, magnesium, and phosphate), buffers (such as HEPES), nucleosides (such as adenosine and thymidine), trace elements (defined as inorganic compounds usually present at final concentrations in the micromolar range), and glucose or an equivalent energy source. In the methods described herein, the medium is preferably supplemented with fetal bovine serum.

In some embodiments, DP cells are cultured under conditions that allow them to self-aggregate, e.g., to adhere to one another to form spheroids. A spheroid is a group of cells in which the majority of cells adheres to other cells and do not adhere to a substrate. A spheroid includes at least two cells and can include, e.g., 4, 8, 12, 20, 40, 50, 100, 500, 1000, or more cells. A spheroid can exist as a cluster of cells suspended in the cell culture medium. Alternatively, the spheroid can be attached. A spheroid is attached when one or more cells of the spheroid are attached to a substrate and the remaining spheroid cells are not directly attached to the substrate but are adhered to those cells of the spheroid that are directly attached to the substrate.

In other embodiments, DP cells are cultured under conditions that allow them to round up: DP cells are “rounded up” when the cells are not attached to a substrate or to another cell. Rounded up cells appear as round balls floating in the cell culture medium. Preferably, at least 70% of the DP cells in a culture are self-aggregated and/or rounded up. Cell culture conditions that allow DP cells to self-aggregate and/or round up can include, e.g., culturing the cells on a non-adherent substrate. Preferably, culturing DP cells on a non-adherent substrate leads to the self-aggregation or rounding up of at least 50%, e.g., at least 70%, of the DP cells. Non-adherent substrates can include, e.g., vessels, e.g., plates, dishes, round-bottom tubes, and flasks, e.g., spinner flasks, that are not treated for tissue culture, e.g., are not treated with, e.g., poly-lysine or collagen. Such vessels are commercially available (e.g., from Corning Life Sciences). Alternatively, non-adherent substrates can include cell culture vessels that are treated with a non-adherent compound, e.g., poly-HEMA. Other non-adherent compounds are known in the art. For example, polymer and copolymer films with hydrophobic components can be used. One form of combination polymer is PEO-PMMA (polyethylene oxide is PEO and PEO-polymethyl methacrylate is PMMA). PMMA can be used as a hydrophobic backbone for PEO oligomer side chains (e.g. 1 PEO: 5 PMMA backbone units) to form a stable water-insoluble film that is resistant to cell adhesion. In still other embodiments, a hydrogel is used, e.g., a neutrally charged hydrogel. For example, the CORNING ULTRA LOW ATTACHMENT SURFACE™ is a covalently bound hydrogel layer that is hydrophilic and neutrally charged and that is non-adherent.

In other embodiments, DP cells are first cultured as an expansion culture on an adherent substrate and then cultured under conditions that allow the DP cells to self-aggregate and/or round up as described herein. Suitable adherent substrates can be, e.g., tissue culture treated vessels, e.g., collagen treated vessels and poly-lysine treated vessels, as described in Culture of Animal Cells: A manual of basic techniques (3^{rd} edition), 1994, R. I. Freshney (ed.), Wiley-Liss, Inc.; Cells: A laboratory manual (vol. 1), 1998, D. L. Spector, R. D. Goldman, L. A. Leinwand (eds.), Cold Spring Harbor Laboratory Press; and Animal Cells: Culture and media, 1994, D. C. Darling, S. J. Morgan, John Wiley and Sons, Ltd. The DP cells are first cultured as an expansion culture to expand the number of DP cells to a desired number, e.g., a number suitable for implantation. The expansion culture can be maintained for at least one day, e.g., at least 2, 5, 7, 10, 14, or 21 days. Alternatively, the expansion culture can be immortalized using routine techniques and maintained indefinitely, as described, e.g., by Lichti et al. (1993) J. Invest. Dermatol. 101:124S-129S.

Methods of Stimulating Hair Growth

The invention also provides methods of stimulating hair growth in which cultured dermal papilla cells with hair-inductive ability are transplanted into a subject, e.g., into the skin of the subject, e.g., into a hair follicle.

In some embodiments, cultured DP cells described herein are subjected to physical and/or biochemical aggregation in order to induce and/or maintain aggregation of the cultured DP cells within the transplantation site. For example, the cultured DP cells can be aggregated through centrifugation of the culture. In addition there can be added to the cultured DP cells, either prior to or at the time of transplantation into the subject, a suitable aggregation enhancing substance, e.g., a glycoprotein such as fibronectin or glycosaminoglycans, e.g., dermatan sulphate, chondroitin sulphates, proteoglycans, heparan sulphate; other extracellular matrix components known to bind DP cells, e.g., collagens; hormones; and growth factors known to induce aggregation.

In other embodiments, the cultured DP cells described herein can be used in a method of screening or evaluating one or more test compounds, e.g., a library of test compounds (e.g., a small molecule or protein compound). The DP cells can be cultured in the presence of a test compound. The cultured cells can be evaluated for a biological parameter, e.g., a parameter related to hair inductive ability measured, ability to transcribe a gene or to produce a protein. For example, the parameter can be gene expression, e.g., expression of the versican promoter, e.g., a reporter fusion thereto. Compounds can be screened for both stimulatory and or repressive effects on parameters related to DP cell hair stimulation. The cultured DP cells can also be used to screen compounds for effects on hair growth following transplantation. The cultured DP cells can be contacted with a test compound during culture or before, during, or after transplantation to a subject, e.g., an animal. Hair growth can then be monitored to determine the effects of the test compound.

In another embodiment, cultured DP cells are used to evaluate the safety or efficacy of a compound, e.g., an aliquot of a product, e.g., a cosmetic or a pharmaceutical product.

The cultured DP cells can be transplanted into a subject using routine methods, e.g., those described by Jahoda et al. (1984) Nature 311:560-562. Various routes of administration and various sites can be used. For example, the cultured DP cells can be introduced directly between the dermis and the epidermis of the outer skin layer at a treatment site. This can be effected by raising a blister on the skin at the treatment site and introducing the cultured DP cells inside the blister, i.e., into the cavity occupied by the blister fluid. The blister may be raised by routine techniques, e.g., by mechanical means such as the application of a reduced pressure suction to the skin, or by chemical means.

The cultured DP cells can also be introduced into a suitable incision extending through the epidermis down into the dermis. The incision can be made using routine techniques, e.g., using a scalpel or hypodermic needle. The incision may be filled with cultured DP cells generally up to a level in direct proximity to the epidermis at either side of the incision.

Alternatively, the cultured DP cells can be introduced into a hair follicle. The hair follicle can first be exposed by a suitable incision prior to introduction of the cultured DP cells. Alternatively, the hair follicle can be filled with the cultured DP cells without first exposing it using, e.g., a hypodermic needle or other suitable delivery device.

The cultured DP cells can also be introduced into the subject together with a different type of cell, e.g., a cultured epidermal cell. The epidermal cell can be derived from the subject, from another subject, or from commercial sources. The cultured epidermal cell can either be in the form of a free epidermal cell or in the form of a sheet of epidermal cells. In the latter case, the cultured DP cells can be enclosed in a pocket formed from the sheet of epidermal cells. Where the cultured DP cells are introduced inside a pocket formed of epidermal cells, the incision in the skin at the treatment site can be formed so as to extend obliquely at a more or less shallow angle to the surface of the skin so as to form in effect a flap of skin under which the pocket of cultured dermal papilla cells can be introduced, the flap then being positioned back over the top of the pocket to seal it in and protect it from external contamination.

In other embodiments, a large plurality of small closely spaced openings is formed in the skin into which the cultured DP cells are transplanted. Each opening can be filled with a large plurality of cultured DP cells. The size and depth of the openings can be varied. The lateral extent of individual openings can be minimized, e.g., limited to about 5 mm, e.g., to about 2 mm. The depth of the openings can be greater than the full depth of the epidermis, e.g., extends at least 1 mm, e.g., at least 3 mm into the dermis.

The openings in the skin can be formed by routine techniques and can include the use of a skin cutting instrument, e.g., a scalpel or a hypodermic needle. Alternatively, a multiple-perforation apparatus can be used having a plurality of spaced cutting edges formed and arranged for simultaneously forming a plurality of spaced openings in the skin. The cultured DP cells can be introduced simultaneously into a plurality, preferably at least several, openings in the skin.

The number of cells introduced into each opening can vary depending on various factors, e.g., the size and depth of the opening and the overall viability and activity of the cells. The number of cells introduced can be, e.g., 1000 to 1,000,000 cells, e.g., 10,000 to 200,000 cells per opening in a volume of, e.g., 0.5 to 50 μL.

In some embodiments, the subject is treated, topically and/or systematically, with a hair growth promoting substance before, at the same time as, and/or after the transplantation of cultured DP cells to enhance hair growth. Suitable hair growth promoting substances can include, e.g., minoxidil (available from the Upjohn Co. of Kalamazoo USA), cyclosporin, and natural or synthetic steroid hormones and their enhancers and antagonists, e.g., anti-androgens.

The cultured DP cells transplanted into a subject can be derived from DP cells taken from the subject. In some embodiments, DP cells are obtained from another subject of the same species or from a different species. Use of cultured DP cells derived from another subject may require administration of an immunosuppressant, alteration of histocompatibility antigens, or use of a barrier device to prevent rejection of the implanted cells.

Cultured DP cells can be administered alone or in conjunction with a barrier or agent for inhibiting immune responses against the transplanted DP cells in a recipient subject. For example, an immunosuppressive agent can be administered to a subject to inhibit or interfere with normal response in the subject. The immunosuppressive agent can be an immunosuppressive drug that inhibits T cell/or B cell activity in the subject. Examples of immunosuppressive drugs are commercially available (e.g., cyclosporin A from Sandoz Corp. East Hanover, N.J.).

An immunosuppressive agent, e.g., drug, can be administered to a subject at a dosage sufficient to achieve the desired therapeutic effect (e.g., inhibition of rejection of the cells). Suitable dosage ranges for immunosuppressive drugs can include, e.g., those described in Freed et al. (1992) N. Engl. J. Med. 327:1549; Spencer et al. (1992) N. Engl. J. Med. 327:1541; and Widner et al. (1992) N. Engl. J. Med. 327:1556. Dosage values may vary according to factors such as age, sex, and weight of the subject.

The immunosuppressive agent can also be an antibody, an antibody fragment, or an antibody derivative that inhibits T cell activity in the subject. Antibodies capable of depleting or sequestering T cells can be, e.g., polyclonal antisera, e.g., anti-lymphocyte serum; and monoclonal antibodies; e.g., monoclonal antibodies that bind to CD2, CD3, CD4, CD8 or CD40 on the T cell surface. Such antibodies are commercially available, e.g., from American Type Culture Collection, e.g., OKT3 (ATCC CRL 8001). An antibody can be administered for an appropriate time, e.g., at least 7 days, e.g., at least 10 days, e.g., at least 30 days, to inhibit rejection of cultured DP cells following transplantation. Antibodies can be administered intravenously in a pharmaceutically acceptable carrier, e.g., saline solution.

Kits for Stimulating Hair Growth

The invention also provides pharmaceutical compositions and kits, e.g., for stimulating hair growth in a subject. A pharmaceutical composition can include DP cells, e.g., cells cultured as described herein, combined with a pharmaceutically acceptable carrier, e.g., saline. The composition can also include an aggregation enhancing substance described herein. In some embodiments, the pharmaceutical composition is packaged within a suitable transplantation apparatus described herein, e.g., a syringe, for direct transplantation into a subject.

In other embodiments, a pharmaceutical composition of cultured DP cells described herein is provided in a solution with instructions for transplanting the composition into a subject. A transplant apparatus described herein can also be included. In some embodiments, a kit including a suitable culture vessel and instructions for performing the methods described herein is provided.

### EXAMPLES

### Example 1

### Isolation of DP Cells

A sharp instrument, such as a pair of scissors, is used to cut a strip of scalp tissue approximately 3 mm wide and 30 mm long from a human male adult subject (obtained, e.g., by a cosmetic surgeon). The white epidermal layer is separated from the underlying fat layer by cutting between the two layers with a pair of toothed scissors. The fat layer containing the lower part of the anagen follicles is used for dissection. The epidermal layer is discarded. The epidermal layer should be completely removed so that the follicles can be pulled out intact.

To separate the follicles cleanly from the fat layer, one pair of forceps is used to hold the fat layer in place. A second pair of forceps is used to pull the anterior end of the follicle to release the follicle intact from the fat layer. The plucked follicles are placed in DMEM culture medium.

### Example 2

### Culture of DP Cells

Dermal papillae from mice expressing GFP under control of the versican promoter (a promoter known to be expressed in dermal papilla cells) (Kishimoto (2000) Genes Dev. 14(10):1181-5) were removed from the hair follicle and surrounding skin, and plated onto a plastic dish containing DMEM medium plus 10% FBS plus antibiotics. DP cells were allowed to migrate out onto the dish from the dermal papilla culture for 2-3 weeks. The DP cells were then trypsinized, and the trypsinized cells were separated by filter from the remaining ball of cells. The trypsinized cells were replated and expanded for 2-3 weeks. During this time, the cells, as expected, lost the ability to express GFP under the versican promoter. After the expansion period, the DP cells were harvested and placed into commercially available well plates with round bottoms and non-adherent sides, which allowed the DP cells to adhere to themselves. After 12 hours, the DP cells regained GFP expression.

### Example 3

### Hair Inductive Ability of DP Cells

The art-recognized nude mouse graft model for testing the hair-forming ability of selected cell populations was used to test the hair-inducing ability of DP cells cultured as in Example 2. DP cells that had regained GFP expression were implanted onto a mouse skin. 30% of the implantations resulted in hair growth compared to none of the control implantations.

### Example 4

### Hair Inductive Ability of DP Cells in Humans

Human dermal papilla cells are collected and cultured using substantially the same procedure as described in Examples 1 and 2 except that the dermal papilla cells are obtained by teasing out dermal papillae using tungsten needles from exposed hair follicles in hair-growing-scalp biopsies and the autologous human serum is used in the growth of cells. Further details of suitable procedures are described in Messenger (1984) Br. J. Derm. 110:685-689.

Prior to transplantation, the cultured DP cells are centrifuged and the pellet is resuspended in 50 μL of saline. Multiple implantation sites are prepared in the skin of the scalp of the subject using a scalpel. The cultured DP cells are introduced into each transplantation site using a hypodermic needle. 50,000 cultured DP cells are implanted into each opening.

The data described herein show that the hair-inductive ability of DP cells that have lost such ability or are at least partially impaired can be reconstituted using the methods of culturing cells described herein. The methods described herein will be useful in methods of inducing hair growth, e.g., in humans.

All patents and references cited herein are hereby incorporated by reference in their entirety. It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of culturing dermal papilla (DP) cells, the method comprising culturing DP cells on a non-adherent substrate.

2-4. (canceled)

5. The method of claim 1, wherein the DP cells are self-aggregated or rounded up.

6. The method of claim 1, wherein the DP cells are cultured for at least 6 hours.

7. The method of claim 1, wherein the DP cells are human DP cells.

8-15. (canceled)

16. A method of culturing DP cells, the method comprising:
(a) expanding DP cells on an adherent substrate; and
(b) thereafter culturing at least a subset of the expanded DP cells under conditions that allow the cells to increase hair inductive activity.

17. The method of claim 16, wherein the adherent substrate is a cell culture plate.

18. The method of claim 16, wherein the adherent substrate is a cell culture flask.

19. The method of claim 16, wherein, in (b), the subset of the cells are cultured on a non-adherent substrate.

20. The method of claim 19, wherein the non-adherent substrate is (a) an untreated cell culture vessel, (b) a culture vessel treated with a non-adherent compound, or (c) a spinner flask.

21. The method of claim 16, wherein the DP cells are expanded for at least 2 days.

22. The method of claim 16, wherein the expanded DP cells are cultured for at least 24 hours.

24. The method of claim 16, wherein the DP cells are human DP cells.

25-30. (canceled)

31. A cell culture comprising DP cells having hair inductive activity, wherein the cells are grown on a non-adherent substrate selected from the group consisting of an untreated culture vessel, a substrate treated with a non-adherent compound, and a spinner flask.

32. The cell culture of claim 31, wherein the DP cells are human DP cells.

33-34. (canceled)

35. The method of claim 1 further comprising transplanting a plurality of the cultured DP cells into the subject.

36. The method of claim 35, wherein the subject is a human subject.

37. The method of claim 35, wherein the DP cells are autologous to the subject.

38. The method of claim 36, wherein the DP cells are allogeneic to the subject.

39. The method of claim 35, wherein the cells are cultured for at least 24 hours.

40. The method of claim 35, wherein the transplanting comprises transplanting the plurality of the cultured DP cells into a hair follicle in the subject's skin.

41-45. (canceled)
